SEQUENCE LISTING (2 sequences)

SEQ ID NO: 1 - protein, 27 aa, Ovis sp.
HSDGIFTDSYSRYRKQMAVKKYLAAVL

SEQ ID NO: 2 - protein, 38 aa, Ovis sp.
HSDGIFTDSYSRYRKQMAVKKYLAAVLGKRYKQRVKNK

(19) United States
(12) Patent Application Publication
(10) Pub. No.: US 2006/0247166 A1
(43) Pub. Date: Nov. 2, 2006

(54) Eye disease treating agent and method for treating eye disease

(75) Inventors: Seiji Shioda, Komae-shi (JP); Tamotsu Seki, Ota-ku (JP); Yuko Shinohara, Okazaki-shi (JP); Masayoshi Nakatani, Nukata-gun (JP); Chisato Taki, Gamagori-shi (JP); Shigeru Nishimura, Kashiwa-shi (JP)

Correspondence Address: SUGHRUE MION, PLLC, 2100 PENNSYLVANIA AVENUE, N.W., SUITE 800, WASHINGTON, DC, 20037, US

(73) Assignee: NIDEK CO., LTD.

(21) Appl. No.: 11/357,100

(22) Filed: Feb. 21, 2006

(30) Foreign Application Priority Data
Apr. 27, 2005 (JP) P. 2005-130609

(51) Int. Cl. A61K 38/17

(52) U.S. Cl. 514/12

(57) ABSTRACT

An eye disease treating agent and a method for treating an eye disease, which use a compound having an interleukin 6 production accelerating activity, are provided.

### FIELD OF THE INVENTION

This invention relates to an eye disease treating agent which inhibits retinal cell death or promotes protection of the retinal cells from cell death, and a method for treating an eye disease.

### BACKGRAOUND OF THE INVENTION

Retinal degenerative diseases (retinal degeneration) are greatly varied depending on their causes and onset modes. As typical examples of the retinal degenerative diseases due to systemic diseases, diabetic retinopathy and hypertensive retinopathy correspond thereto. For the treatment of these diseases, application of causal treatment such as administration of a hypoglycemic agent or hypotensive agent is mostly carried out, but retinal ganglion cell death cannot always be prevented or treated by this. In addition to this, retinal artery occlusion, retinal vein occlusion, retinopathy of prematurity and the like are included in the diseases in which a retinal blood vessel lesion is the main symptom, but there are no decisive therapeutic agents for these cases, and it is the present situation that they are relying upon operative treatments. On the other hand, it is considered that retinal ganglion cell death is deeply concerned also in the onset of macular degeneration, pigmentary retinal dystrophy and the like other retinal degenerative diseases.

On the other hand, in the treatment of cerebral diseases, it has been shown that a peptide hormone called PACAP (pituitary adenylate cyclase-activating polypeptide), which is broadly distributed in the central nerves and peripheral tissues and concerned in the differentiation and survival of neuronal cells, activation of nervous secretory system and the like, inhibits cranial neuronal cell death, and this is now a candidate of cerebral disease treatment (cf. Patent Reference 1: JP-T-10-505863 (the term “JP-T” as used herein means a published Japanese translation of a PCT patent application)).

### SUMMARY OF THE INVENTION

However, there is no minute examination on the kind of effect of PACAP upon retinal neuronal cells. Accordingly, the object of invention is to provide an eye disease treating agent which is suited for the prevention and treatment of retinal neuronal cells in eye diseases, and a method for treating an eye disease.

The present inventors have conducted intensive studies using various experimental models and found as a result that production of an endogenous multifunctional cytokine IL-6 (interleukin 6) is accelerated in Muller cell which is a main glial cell (neuroglia cell) of the retina, by the administration of PACAP, and retinal ganglion cell death is thereby indirectly inhibited or delayed. The constitution of the invention is set forth below.

1. An eye disease treating agent, which comprises a compound having an interleukin 6 production accelerating activity as an active ingredient.

2. The eye disease treating agent according to the item 1, wherein the compound having an interleukin 6 production accelerating activity is pituitary adenylate cyclase-activating polypeptide (PACAP) or a PACAP analog.

3. The eye disease treating agent according to the item 1, wherein the compound having an interleukin 6 production accelerating activity is a PAC 1 receptor agonist.

4. The eye disease treating agent according to any one of the items 1 to 3, wherein the eye disease is a retinal disease.

5. The eye disease treating agent according to any one of the items 1 to 4, which is an oral administration agent.

6. The eye disease treating agent according to any one of the items 1 to 4, which is an ophthalmic solution.

7. The eye disease treating agent according to any one of the items 1 to 4, which is an ophthalmic ointment.

8. The eye disease treating agent according to any one of the items 1 to 4, which is a nasal drop.

9. The eye disease treating agent according to any one of the items 1 to 4, which is an injection.

10. A method for treating an eye disease, which comprises administering to a subject in need thereof an effective amount of a compound having an interleukin 6 production accelerating activity.

11. The method according to the item 10, wherein the compound having an interleukin 6 production accelerating activity is pituitary adenylate cyclase-activating polypeptide (PACAP) or a PACAP analog.

12. The method according to the item 10, wherein the compound having an interleukin 6 production accelerating activity is a PAC 1 receptor agonist.

13. The method according to any one of the items 10 to 12, wherein the eye disease is a retinal disease.

14. The method according to any one of the items 10 to 13, wherein the compound having an interleukin 6 production accelerating activity is administered as an oral administration agent.

15. The method according to any one of the items 10 to 13, wherein the compound having an interleukin 6 production accelerating activity is administered as an ophthalmic solution.

16. The method according to any one of the items 10 to 13, wherein the compound having an interleukin 6 production accelerating activity is administered as an ophthalmic ointment.

17. The method according to any one of the items 10 to 13, wherein the compound having an interleukin 6 production accelerating activity is administered as a nasal drop.

18. The method according to any one of the items 10 to 13, wherein the compound having an interleukin 6 production accelerating activity is administered as an injection.

19. The method according to claim 11, wherein by the administration of PACAP or PACAP analog, a production of IL-6 in Muller cell is accelerated to indirectly inhibit or delay retinal ganglion cell death.

Since the eye disease treating agent of the invention has the effect to inhibit retinal ganglion cell death via retinal Muller cell, it can be used as an agent effective for the prevention and treatment of optic neuronal diseases such as glaucoma, retinal degenerative diseases due to systemic diseases such as diabetic retinopathy, hypertensive retinopathy and systemic lupus erythematosus, retinal blood vessel occlusion and lesional diseases such as retinopathy of prematurity, retinal vein occlusion, retinal artery occlusion, retinal periphlebitis occlusion, inflammations and degenerations caused by retinal detachment or injury, retinal degenerative diseases accompanied by aging such as aging macular degeneration, and the like.

### BRIEF DESCRIPTION OF THE DRAWINGS

FIG. 1 is a graph showing IL-6 production in the culture liquid 24 hours after the addition of PACAP 38 to cultured Muller cell.

FIG. 2 is a graph showing inhibitory effect of a PACAP receptor antagonist upon the IL-6 production acceleration effect of PACAP 38 in cultured Muller cell.

FIG. 3 is a fluorescence microphotograph showing a result of anti-IL-6 immunostaining of the retinal tissue after administration of PACAP 38 into the vitreous body.

FIG. 4 is a graph showing the number of cells remaining in the ganglion cell layer of the retinal tissue after administration of PACAP 38 into the vitreous body, in a kainic acid-induced retinal ganglion cell death model.

FIG. 5 is a graph showing the retinal tissue pNF-H content after pre-administration of PACAP 38 or its simultaneous administration with kainic acid, in a kainic acid-induced retinal ganglion cell death model.

### DETAILED DESCRIPTION OF THE INVENTION

It was confirmed that IL-6 production of Muller cell in the retinal tissue is accelerated in a delaying manner when PACAP is administered into the vitreous body.

The inventors have confirmed that the IL-6 production acceleration effect of PACAP in cultured Muller cells is generated via a specific receptor PAC 1 receptor when a low concentration (10⁻¹² M) of PACAP is added, but via a different receptor at a high concentration (10⁻⁸ M). The PAC 1 receptor belongs to the 1 class of G protein coupling receptor (GPCR) which activates various intracellular signaling pathways. It is known that activation of the PAC 1 receptor in the central nervous system takes an important role in the development, function and survival of the nervous system. Based on this, it was found that PACAP can be used as a therapeutic or preventive agent at such a markedly low concentration that side effects and complications in the retina can be avoid. Accordingly, the invention is characterized in that an effective amount of PACAP which interacts only with the PAC 1 receptor or a PAC 1 receptor agonist is administered.

In the present invention, PACAP is particularly unlimited as far as it has IL-6 production accelerating activity, but PACAP is preferably one capable of interacting with PAC 1 receptor. Specific Examples of PACAP include PACAP27 having amino acid residues of 27 and PACAP38 having amino acid residues of 38.

In addition, PACAP analog can also be used as the active ingredient of the therapeutic agent. The term PACAP analog as used herein means PACAP that is artificially modified by a conventional process and has IL-6 production accelerating activity. The PACAP analog includes a PACAP analog containing an effective group for a solubilization such as a hydrogen atom or a carboxylic acid residue having a carbon atom of 1 to 20 at the N-terminal, or a PACAP analog containing a group such as a hydrogen atom, —NH—, —OH or a carboxyl group having a carbon atom of 1 to 4 at the C-terminal, which have the same activity as PACAP.

The solvate or salts of PACAP or PACAP analog such as a PACAP hydrate can also be used as the active ingredient of the therapeutic agent.

The PACAP can be obtained by a method described in Biochem. Biophys. Res. Commun., 164:567-574 (1989) and Biochem. Biophys. Res. Commun., 170:643-648 (1990). The amino acid sequence of PACAP27 and The amino acid sequence of PACAP38 are shown in SEQ ID NO:1 and SEQ ID NO:1, respectively.

In this connection, in addition, non-peptide agonist for a PACAP receptor capable of accelerating production of IL-6 may also be used as the active ingredient of the therapeutic agent. Particularly, a non-peptide agonist which interacts with PAC 1 receptor is preferable.

When such the compound having IL-6 production accelerating activity (preferably, PACAP) is used as a therapeutic agent for a retinal degenerative disease, it can be administered orally (e.g., tablets, capsules, granules or the like) or parenterally (e.g., ophthalmic solutions, ophthalmic ointments, nasal drops, injections or the like) as various types of pharmaceutical compositions by mixing it with pharmacologically acceptable carriers, fillers, diluents and the like. In the present invention, the compound having IL-6 production accelerating activity (preferably, PACAP) is preferably administered parenterally (e.g., ophthalmic solutions, ophthalmic ointments, nasal drops, injections or the like), more preferably administered topically (ophthalmic solutions and ophthalmic ointments).

For example, in the case of oral administration, tablets containing a predetermined amount of the active ingredient are used. The tablets can be produced, for example, by compressing or forming the compound itself or together with other auxiliary components (a diluent, a binder, a lubricant, a preservative and the like).

As mentioned above, the compound having IL-6 production accelerating activity (preferably, PACAP) may be administered either by oral administration or by parenteral administration. The dose is optionally decided by taking into consideration conditions of each case such as symptoms, age, sex and the like of the patient to be treated, but, usually, it is orally administered within the range of from 2.5×10⁻⁵ mg to 25 mg, preferably from 25×10⁻⁵ mg to 25×10⁻³ mg, per day per adult by dividing the daily dose into 1 to several doses per day, or intravenously injected within the range of from 2.5×10⁻⁶ mg to 2.5 mg, preferably from 25×10⁻⁶ mg to 2.5×10⁻³ mg, per day per adult by dividing the daily dose into 1 to several doses per day, or continuously and intravenously injected within the range of from 1 to 24 hours per day. Also, it is parenterally (ophthalmic solutions and ophthalmic ointments) administered within the range of from 2.5×10⁻⁶ mg to 2.5 mg, preferably from 2.5×10⁻⁶ mg to 2.5×10⁻⁴ mg, per day per a single eye of adult by dividing the daily dose into 1 to several doses per day. As a matter of course, since the dose varies under various conditions as described in the foregoing, a smaller dose than the above range may be sufficient enough in some cases.

In the case of parenteral administration, it is desirable that the amount of PACAP in the composition for administration use is 10,000 times of the effective amount in the retina.

For example, when PACAP is used as ophthalmic solutions, PACAP is added in an amount of from 0.01% to 1% (w/v) to a base material solvent and made into an aqueous solution or a suspension.

Various additive agents may be added to the ophthalmic solutions of the invention, such as buffer agents (e.g., phosphate buffer agents, borate buffer agents, citrate buffer agents, tartarate buffer agents, acetate buffer agents, amino acids and the like), tonicity agents (e.g., sorbitol, glucose, mannitol and the like saccharides, sodium chloride and the like salts and the like), antiseptics (e.g., benzalkonium chloride, benzethonium chloride, parabens, benzyl alcohol and the like), pH adjusting agents (e.g., hydrochloric acid, phosphoric acid, sodium hydroxide and the like), thickeners (e.g., hydroxyethyl cellulose, hydroxypropyl cellulose, methyl cellulose, hydroxypropylmethyl cellulose, carboxymethyl cellulose and salts thereof and the like), solubilizing agents(e.g., ethanol, polyoxyethylene hydrogenated castor oil, Polysorbate 80 and the like) and the like.

In addition, when PACAP is used as ophthalmic ointments, they are produced by mixing approximately from 0.01% tol % (w/v) of PACAP with a general ophthalmic ointment base. As the ophthalmic ointment base, purified lanolin, white petrolatum, macrogol, Plastibase, liquid paraffin and the like are used.

In addition to the components illustratively cited in the above, other drug effect components or other components used in this field (correctives and the like) may be contained in the retinal neuronal and retinal degenerative disease treating agent of the invention.

Test examples of the invention are shown in the following. Effects of the invention are revealed by these examples, but the scope of the invention is not limited thereto.

### TEST EXAMPLE 1

Using cultured rat Muller cell, IL-6 production acceleration effect of PACAP 38 was examined. In this connection, PACAP has two molecular types of PACAP 38 and PACAP 27 respectively having 38 amino acid residues and 27 residues, but the PACAP 38 was used herein.

(Isolation and Culturing of Muller Cell)

Wister/ST rats of 14 days of age (20 animals) were sacrificed by bleeding through the cutting of abdominal aorta under diethyl ether anesthesia. The 40 eyeballs were immediately enucleated and soaked in Dulbecco's modified minimum essential medium (DMEM) containing 25 mM HEPES and antibiotics (penicillin 100 U/ml, streptomycin 100 μg/ml, amphotericin B 0.25 μg/ml), one night at room temperature under shade. Next, the eye balls were transferred into DMEM containing 0.1% trypsin and 70 U/ml collagenase and incubated at 37° C. for 60 minutes. Thereafter, retinas were removed from the eyeballs and transferred into DMEM supplemented with antibiotics and 10% FBS (medium for culturing use) (8 retinas/10 ml DMEM). The retinas were dispersed and suspended until they became small aggregates through the pipetting using a Pasteur pipette having thinned tip. This cell suspension was seeded onto a polystyrene dish (Asahi Techno Glass) and cultured by keeping at 37° C. under an environment of 5% CO₂/95% air. After 5 days of the seeds, the aggregates and cell residues suspended in the culture liquid or adhered to the dish bottom were almost completely removed by carrying out pipetting using fresh medium for culturing use. Thereafter, medium exchange was carried out at a frequency of 3 days a week, and the culturing was continued until the cells became almost confluent state.

(Test Method)

The cells of P2 were used in the test. That is, the Muller cells isolated from retinas and cultured to confluent state were sub-cultured (P 1), and the Muller cells of P1 when became confluent state were used in the test in the following manner.

Muller cells were separated from a dish using trypsin and then seeded into 24 well culture plate (5×10⁴ cells/500 μl/well) and cultured using the medium for culturing use. The medium was exchanged with serum-free DMEM 24 hours thereafter. After continuing the culturing for 24 hours, PACAP 38 (10⁻¹² M, 10⁻⁹ M to 10⁻⁶ M) was added thereto and the culturing was continued for 24 hours. Impurities were precipitated by lightly centrifuging the culture plate, and then 450 μl of the culture supernatant was recovered.

The IL-6 concentration in the thus recovered Muller cell culture supernatant was measured using B9 cell which is a hybridoma sub-clone of an IL-6-dependent mouse. The B9 cell was cultured using a medium prepared by adding. A 2 μl hybridoma growth units/ml of human recombinant IL-6 to RPMI 1640 containing 25 mM HEPES, 10% FBS and 1% antibiotic-antimycotic solution, under an environment of 5% CO₂/95% air and 37° C. A 2 μl portion of each sample was added to each well of a 96 well plate into which an IL-6 un-added medium had been dispensed in 198 μl/well portions, and 8 times-diluted in order. Subsequently, 100 μl of the B9 cell suspension (2 to 4×10⁴ cells/ml) was added to each well and cultured for 72 hours. Regarding the bioassay of IL-6, growth of B9 cell was determined as the fluorescence intensity by Alamar Blue.

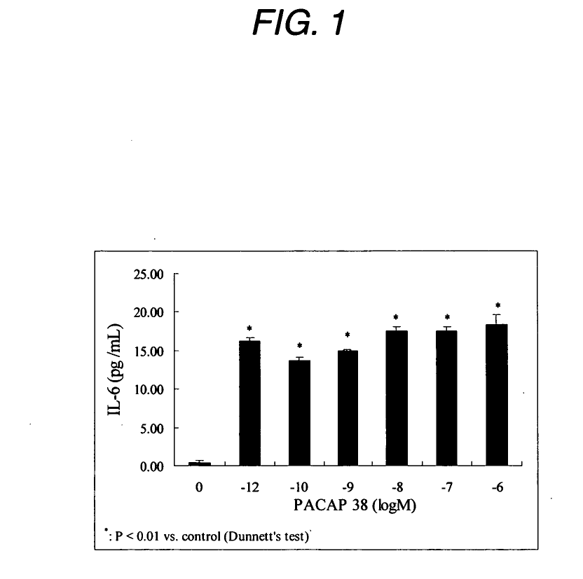

The results are shown in FIG. 1. In the case of the no addition of PACAP, the Muller cell hardly produced IL-6. On the other hand, IL-6 concentration in the culture liquid significantly increased when PACAP was added (p<0.01). What is more, even at a low PACAP 38 adding amount of 10⁻¹² M in concentration, production of IL-6 similar to the case of the addition of 10⁻⁶ M was found. Based on this result, it was revealed that a very small amount (pM, nM order) of PACAP 38 has the IL-6 production acceleration effect in the cultured Muller cell.

### TEST EXAMPLE 2

Examination was carried out un whether or not the IL-6 production acceleration effect in Muller cell is specific to PACAP. In this test, whether or not the IL-6 production acceleration effect by PACAP 38 is inhibited was examined using a competitive PACAP receptor antagonist PACAP 6-38.

(Test Method)

The rat retina derived Muller cell was cultured in the same manner as in Test Example 1. After culturing for 24 hours using the serum-free medium, PACAP 38 (10⁻¹²M or 10⁻⁸ M) or PACAP 6-38 (10⁻⁸ M or 10⁻⁶ M) was added thereto, and the culturing was further carried out for 24 hours. In the same manner as in Test Example 1, the IL-6 concentration was measured by recovering the culture supernatant,

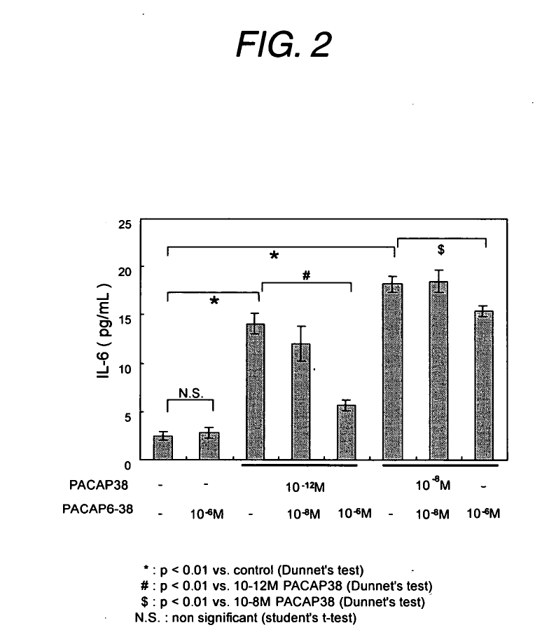

The results are shown in FIG. 2. It was revealed that the IL-6 effect of PACAP 38 is inhibited by PACAP 6-38. In addition, the inhibitory effect was observed more significantly in the low concentration (10⁻¹² M) PACAP 38 addition group.

Based on the above results, it was confirmed that the increase of IL-6 concentration in the Muller cell culture liquid found in Test Example 1 is due to PACAP 38 In addition, it was revealed that PACAP 38 accelerates the IL-6 production via PAC 1 receptor in the case of the addition of 10⁻¹² M, or via a receptor different from PAC 1 in the case of the addition of 10⁻⁸ M.

### TEST EXAMPLE 3

By administering a PACAP 38 solution into the vitreous body, whether or not it is effective in accelerating IL-6 production in the retina tissue was examined.

(Preparation of Administration Liquid)

The PACAP 38 was dissolved in sterile physiological saline to a concentration of 3.3 nmol/ml. This was stored under ice-cooling until just before the administration.

(Test Method)

Male Wister/ST rats of 8 weeks of age were used in this test. After anesthesia by intraperitoneal administration of pentobarbital sodium, 3 μl per one eye of the administration liquid was injected from about 1 mm behind the limbus using a Hamilton glass syringe equipped with a 30 G needle, that is, the liquid was injected into the vitreous body in such a manner that 10⁻¹¹ mol of PACAP reached the retina. After 1, 2, 3 and 7 days of the PACAP administration, each animal was sacrificed by bleeding through the cutting of abdominal aorta under diethyl ether inhalation anesthesia, and then both eyeballs were enucleated, and fixed with ice-cold 4% paraformaldehyde-0.1 M phosphate buffer (pH 7.4). The fixed eyeballs were subjected to sucrose replacement and then freeze-embedded to prepare frozen sections of 10 μm thickness. By applying fluorescence immunostaining to each of the frozen sections using an anti-IL-6 antibody, expression of IL-6 in the retina was observed.

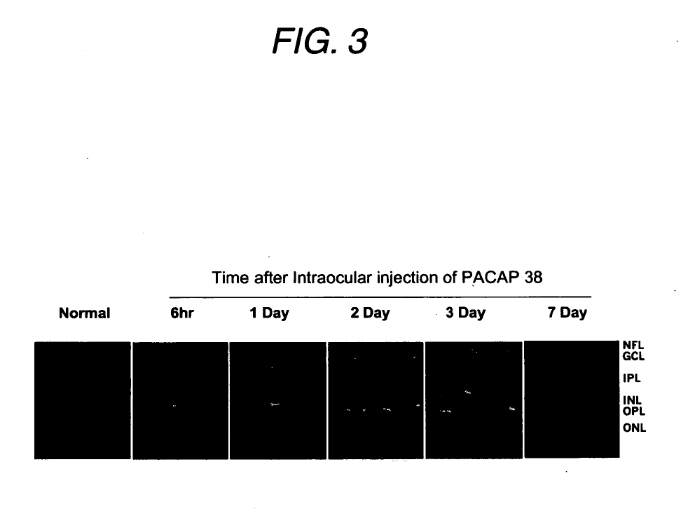

The results are shown in FIG. 3. Expression of IL-6 was not found in the PACAP un-administered eyes and retinas of 1 day after the administration. On the other hand, an IL-6-positive cell which is stained over a region of from the inner limiting membrane to the outer nuclear layer of the retina was frequently observed after 2 days and 3 days of the PACAP administration. Expression of IL-6 was hardly observed after 7 days. It was confirmed from its localization and morphology that this IL-6-positive cell is the Muller cell. Based on the above results, it was revealed that production of IL-6 is accelerated in Muller cell in a delaying manner by the administration of PACAP into vitreous body.

### TEST EXAMPLE 4

Retinal nerve protecting effect was examined using a kainic acid-induced retinal ganglion cell death animal model. This animal model was used, because it shows a morbid state similar to glaucoma (apoptosis of retinal ganglion cell) by inducing glutamate excitotoxicitic neuronal cell death which causes glaucoma.

(Test Method)

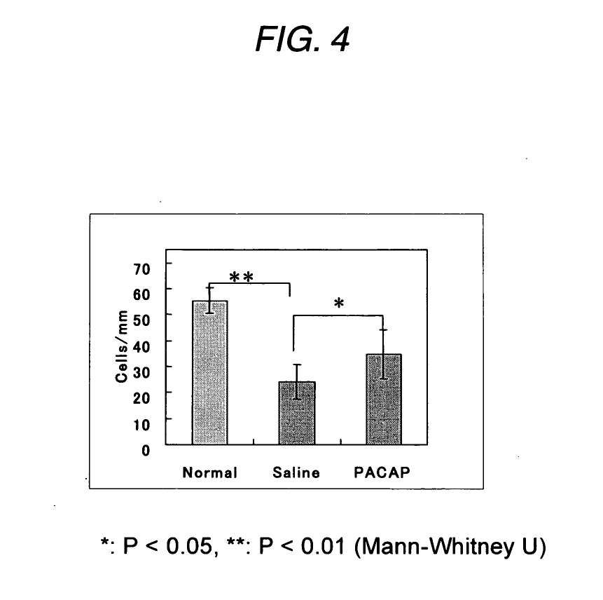

Male Wister/ST rats of 8 weeks of age were used in the test. After anesthesia by intraperitoneal administration of pentobarbital sodium, PACAP 38 (10 pmol) dissolved in sterile physiological saline or the administration solvent (physiological saline) as the negative control was injected once into the vitreous body in the same manner as in Test Example 3. Two days after the PACAP administration, kainic acid (5 nmol) was injected once into the vitreous body of each animal under anesthesia by a mixed liquid of ketamine and seractal. 7 days after the kainic acid administration, all animals were sacrificed by bleeding through the cutting of abdominal aorta under diethyl ether inhalation anesthesia and their eyeballs were enucleated. The enucleated eye balls were fixed by soaking in a 1% formaldehyde-1.25% glutaraldehyde mixed fixing liquid. The fixed eyeballs were embedded in paraffin, and then retinal tissue sections of 4 μm in thickness traversing the optic disc were prepared and subjected to hematoxylin eosin staining. Effect of PACAP was judged by counting the number of cells remaining in the ganglion cell layer within a region of from 0.5 to 1.7 mm from the optic disc of each tissue specimen. The results are shown in FIG. 4. Average value of the number of remaining cells in the control group (n=8) in which physiological saline was administered was 24.0 cells/mm, reducing to 43% of the untreated group. Average value of the number of cells in the PACAP administered group 34.8 cells/mm, so that inhibition of the reduction of the number of cells caused by kainic acid was found with a significance (p<0.05) in comparison with the control group.

### TEST EXAMPLE 5

In order to clarify whether or not the IL-6 which is expressed in a delayed manner in Muller cell (Test Example 3) is concerned in the retinal ganglion cell death inhibitory effect of PACAP confirmed in Test Example 4, this was verified using the same animal model and using neurofilament (phosphorylated neurofilament-H; pNF-H) protein in the retinal tissue as the index. It is known that pNF-H is a protein which is specifically expressed in the retinal ganglion cell, and that the content of this protein in the retinal tissue can be used as a quantitative index for estimating degree of the retinal ganglion cell death.

(Test Method)

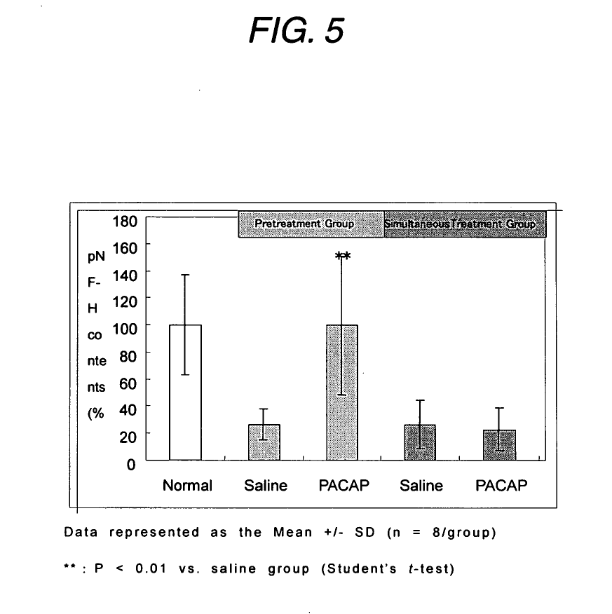

A group in which 10 pmol of PACAP 38 is injected into the vitreous body 2 days before the kainic acid exposure in the same manner as in Test Example 4 (pretreatment group) and another group in which the same amount of PACAP is injected simultaneously with kainic acid (simultaneous treatment group) were set up as the test groups. The retinal tissue was collected 7 days after the kainic acid exposure, and the pNF-H protein was extracted. Amount of the pNF-H protein in retinal tissue was measured by the sandwich ELISA. The results are shown in FIG. 5. In the PACAP 38 pretreatment group, reduction of the amount of pNF-H protein in the retinal tissue induced by the kainic acid exposure was inhibited with a significance (p<0.01) in comparison with the physiological saline group used as the control. On the other hand, the effect to inhibit reduction of the amount of pNF-H was completely absent in the PACAP 38 simultaneous treatment group. This result supports that the IL-6 expressing in a delayed manner in Muller cell is concerned in the retinal neuroprotective effect of PACAP.

As described in the above, it was shown by both of the in vitro and in vivo test examples that an extremely small amount of PACAP accelerates production of a nerve protecting factor IL-6 via PAC 1 receptor in Muller cell which is a glial cell having an important role in the construction and function of the retina, and thereby contributes to the protection of retinal ganglion cell death. Since the preventive and therapeutic agents of the invention have the effect to inhibit retinal ganglion cell death via retinal Muller cell, they can be used as agents effective for the prevention and treatment of optic neuronal diseases such as glaucoma, retinal degenerative diseases due to systemic diseases such as diabetic retinopathy, hypertensive retinopathy and systemic lupus erythematosus, retinal blood vessel lesional diseases such as retinopathy of prematurity, retinal vein occlusion, retinal artery occlusion, and retinal periphlebitis, inflammations and degenerations caused by retinal detachment or injury, retinal degenerative diseases accompanied by aging, aging macular degeneration, and the like. In this connection, examinations were made on PACAP 38 in the test examples described in the foregoing, but the same can also be applied to PACAP 27.

This application is based on Japanese patent application JP 2005-130609, filed on Apr. 27, 2005, the entire content of which is hereby incorporated by reference, the same as if set forth at length.

What is claimed is:

1. An eye disease treating agent, which comprises a compound having an interleukin 6 production accelerating activity as an active ingredient.

2. The eye disease treating agent according to claim 1, wherein the compound having an interleukin 6 production accelerating activity is pituitary adenylate cyclase-activating polypeptide (PACAP) or a PACAP analog.

3. The eye disease treating agent according to claim 1, wherein the compound having an interleukin 6 production accelerating activity is a PAC 1 receptor agonist.

4. The eye disease treating agent according to Claim 1, wherein the eye disease is a retinal disease.

5. The eye disease treating agent according to claim 1, which is an oral administration agent.

6. The eye disease treating agent according to claim 1, which is an ophthalmic solution.

7. The eye disease treating agent according to claim 1, which is an ophthalmic ointment.

8. The eye disease treating agent according to claim 1, which is a nasal drop.

9. The eye disease treating agent according to claim 1, which is an injection.

10. A method for treating an eye disease, which comprises administering to a subject in need thereof an effective amount of a compound having an interleukin 6 production accelerating activity.

11. The method according to claim 10, wherein the compound having an interleukin 6 production accelerating activity is pituitary adenylate cyclase-activating polypeptide (PACAP) or a PACAP analog.

12. The method according to claim 10, wherein the compound having an interleukin 6 production accelerating activity is a PAC 1 receptor agonist.

13. The method according to claim 10, wherein the eye disease is a retinal disease.

14. The method according to claim 10, wherein the compound having an interleukin 6 production accelerating activity is administered as an oral administration agent.

15. The method according to claim 10, wherein the compound having an interleukin 6 production accelerating activity is administered as an ophthalmic solution.

16. The method according to claim 10, wherein the compound having an interleukin 6 production accelerating activity is administered as an ophthalmic ointment.

17. The method according to claim 10, wherein the compound having an interleukin 6 production accelerating activity is administered as a nasal drop.

18. The method according to claim 10, wherein the compound having an interleukin 6 production accelerating activity is administered as an injection.

19. The method according to claim 11, wherein by the administration of PACAP or PACAP analog, a production of IL-6 in Muller cell is accelerated to indirectly inhibit or delay retinal ganglion cell death.
